(19) United States
(12) Patent Application Publication
(10) Pub. No.: US 2009/0124632 A1
(43) Pub. Date: May 14, 2009

(54) Methods and kits related to the topical administration of quinolone antibiotics°

(76) Inventor: Reza Babapour, Beverly Hills, CA (US)

Correspondence Address: 212° Consulting, Inc.;Bungalow B, 851 Moraga Road, Lafayette, CA, 94549, US

(21) Appl. No.: 12/291,601

(22) Filed: Nov. 12, 2008

Related U.S. Application Data
(60) Provisional application No. 61/003,032, filed on Nov. 13, 2007.

(51) Int. Cl. A61K 31/496; A61P 31/04; A61P 31/10

(52) U.S. Cl. 514/253.08

(57) ABSTRACT

The present invention is generally directed to the treatment of dermatological bacterial infections, burns and wounds. It is more specifically directed to the treatment of such infections, burns and wounds using one or more quinolone antibiotics. In one method of treatment aspect, an aqueous solution containing Ciprofloxacin-HCl is topically applied to the skin of a patient exhibiting a dermatological disorder. In another method of treatment aspect, an ointment containing Ciprofloxacin-HCl is topically applied to the skin of a patient exhibiting a dermatological disorder.

### CROSS-REFERENCE TO RELATED APPLICATIONS

This application claims priority to U.S. Provisional Patent Application Ser. No. 61/003,032 filed on Nov. 13, 2007, the entire disclosure of which is incorporated by reference.

### FIELD OF THE INVENTION

The present invention is generally directed to the treatment of dermatological bacterial infections, burns and wounds. It is more specifically directed to the treatment of such infections, burns and wounds using one or more quinolone antibiotics.

### BACKGROUND OF THE INVENTION

Quinolone antibiotics have been used for the treatment of particular bacterial infections for a number of years. Systemically, they have been administered for urinary-tract infections, prostatitis, sexually transmitted diseases, gastrointestinal and abdominal infections, respiratory tract infections, and bone and soft tissue infections. Topically, they are used to treat ophthalmic and otic infections.

Relative to traditional systemic, ophthalmic and otic use, there is little information on the application of quinolone antibiotics to the topical treatment of dermatological disorders.

U.S. Pat. No. 6,309,669, for instance, discusses systemic treatment with anti-inflammatories (e.g., Ciprofloxacin) for the prevention of wart re-occurrence.

U.S. Pat. No. 6,017,912 reports a topical formulation containing a fluoroquinolone mixed with acetone and alcohol, where the formulation is for the treatment of skin bacterial infections. Applicants believe this formulation was proposed as a way to circumvent the low water solubility of fluoroquinolones.

U.S. Pat. No. 5,912,255 reports a topical formulation containing a fluoroquinolone and benzoyl peroxide for the treatment of skin disorders, especially acne. The presented formulation is an ointment, and contains alcohol, petrolatum and emulsifying agents. The benzoyl peroxide allegedly promotes the fluoroquinolone activity, which is implicitly insufficient.

Applicants firmly believe that previous work directed to fluoroquinolone formulations and combinations with other active agents has been directed to reducing the amount of fluoroquinolone one might have to topically apply for a skin disorder treatment. That is because fluoroquinolones are known to produce adverse skin reactions in certain individuals, including sensitivity, rash, and lesions.

### SUMMARY OF THE INVENTION

The present invention is generally directed to the treatment of dermatological bacterial infections, burns and wounds. It is more specifically directed to the treatment of such infections, burns and wounds using one or more quinolone antibiotics.

In a method of treatment aspect, an aqueous solution containing Ciprofloxacin-HCl is topically applied to the skin of a patient exhibiting a dermatological bacterial infection, a burn or a wound.

The concentration of Ciprofloxacin-HCl in the aqueous formulation typically ranges from 2.0 mg/mL solution to 5.0 mg/mL solution. It preferably ranges from 2.5 mg/mL to 4.5 mg/mL or 3.0 mg/mL to 4.0 mg/mL. More preferably, it is about 3.5 mg/mL.

The solution optionally contains a preservative, which is preferably benzalkonium chloride. Where it is included the concentration of preservative in the solution typically ranges from 0.001% to 0.010%. Preferably it ranges from 0.004% to 0.008%, and more preferably it is about 0.006%.

The solution furthermore optionally contains nonmedicinal ingredients. Nonlimiting examples of such ingredients include: acetic acid; edetate disodium; hydrochloric acid; sodium hydroxide; mannitol; purified water; and, sodium acetate.

In another method of treatment aspect, an ointment containing Ciprofloxacin-HCl is topically applied to the skin of a patient exhibiting a dermatological bacterial infection, a burn or a wound.

The concentration of Ciprofloxacin-HCl in the ointment typically ranges from 2.0 mg/g of ointment to 5.0 mg/g of ointment. It preferably ranges from 2.5 mg/g to 4.5 mg/g or 3.0 mg/g to 4.0 mg/g. More preferably it is about 3.5 mg/g.

The ointment optionally contains mineral oil and/or white petrolatum.

In another method of treatment aspect, either the aqueous solution or ointment described above is applied topically to a dermatological bacterial infection, a burn or a wound for at least three (3) days. Preferably, it is applied for at least five (5) or seven (7) days. More preferably, it is applied for at least ten (10) or twelve (12) days. Most preferably, it is applied for fourteen (14) days.

The amount of solution applied per application typically ranges from 0.01 mL to 1.0 mL. Preferably, the amount applied ranges from 0.05 mL to 0.90 mL or 0.1 mL to 0.80 mL. More preferably, the amount applied ranges from 0.2 mL to 0.7 mL.

The amount of ointment applied per application typically ranges from 0.01 g to 1.0 g. Preferably, the amount applied ranges from 0.05 g to 0.90 g or 0.1 g to 0.80 g. More preferably, the amount applied ranges from 0.2 g to 0.7 g.

The number of applications per day of either the solution or the ointment to the skin disorder are typically one (1) or two (2).

In a kit aspect, either the solution or ointment described above is included in a container. The kit further includes instructions on the use of the solution or ointment, which typically contain one or more of the following: the amount of solution or ointment used per application; the number of applications per day; and, the number of days for which the solution or ointment should be applied.

In another method of treatment aspect, either an aqueous solution containing Ciprofloxacin-HCl or an ointment containing Ciprofloxacin-HCl—as described above—is sequentially applied topically to the skin of a patient exhibiting a dermatological bacterial infection, burn or wound with a formulation containing one or more imidazole and/or triazole antifungal agents.

In another kit aspect, either the solution or ointment described above is included in a container. The kit further includes instructions on the use of the solution or ointment along with instructions on the sequential administration of a formulation containing one or more imidazole and/or triazole antifungal agents. The instructions typically include: the amount of Ciprofloxacin-HCl containing solution or ointment to be used; the amount of imidazole and/or triazole antifungal agent containing formulation to be used; the number of sequential applications per day; and, the number of days for which the formulations should be sequentially applied.

### DETAILED DESCRIPTION OF THE INVENTION

Definitions

“Quinolone Antibiotic” refers to a class of antibiotics that are 4-quinolones and contain a carboxylic acid moiety in the 3 position of the basic ring structure. Non-limiting examples of Quinolone Antibiotics include: Nalidixic Acid; Cinoxacin; Norfloxacin; Ciprofloxacin; Ofloxacin; Sparfloxacin; Lomefloxacin; Fleroxacin; Perfloxacin; and, Amifloxacin.

“Dermatological Bacterial Infection” refers to the colonization of bacteria in a particular location within the skin or nails. Nonlimiting examples of such infections include: Folliculitis; Furuncles; Carbuncles; Erysipelas; Hydradenitis Supperativa; skin abcesses; Cellulitis; Paronychia; nail infection (e.g., pseudomonas) and, Impetigo. Nonlimiting examples of bacteria that can infect skin include: Staphylococcus; Pneumococcus; Streptococcus; and, Corynebacterium minutissimum.

“Imidazole Antifungal Agent” refers to an antifungal agent that contains an imidazole moiety. Nonlimiting examples of imidazole antifungal agents include: Clotrimazole; Econazole; Miconazole; Terconazole; and, Butoconazole.

“Triazole Antifungal Agent” refers to an antifungal agent that contains a triazole moiety. Nonlimiting examples of triazole antifungal agents include: Ketoconazole; Itraconazole; and, Fluconazole.

The present invention is generally directed to the treatment of dermatological bacterial infections, burns and wounds. It is more specifically directed to the treatment of such infections, burns and wounds using one or more quinolone antibiotics.

The quinolone antibiotics used in the present invention (e.g., Ciprofloxacin-HCl) are typically formulated as aqueous solutions or ointments.

Where the antibiotic is formulated as an aqueous solution, the quinolone antibiotic is typically present at a concentration ranging from 2.0 mg/mL to 5.0 mg/mL. The concentration preferably ranges from 2.5 mg/mL to 4.5 mg/mL or 3.0 mg/mL to 4.0 mg/mL. More preferably, it is about 3.5 mg/mL.

The aqueous solution optionally contains a preservative, which is typically benzalkonium chloride. Where it is included the concentration of preservative in the solution typically ranges from 0.001% to 0.010%. Preferably it ranges from 0.004% to 0.008%, and more preferably it is about 0.006%.

Nonmedical ingredients are oftentimes included in the aqueous solution. Nonlimiting examples of such ingredients include: acetic acid; edetate disodium; hydrochloric acid; sodium hydroxide; mannitol; purified water; and, sodium acetate.

Where the antibiotic is formulated as an ointment, the quinolone antibiotic is typically present at a concentration ranging from 2.0 mg/g of ointment to 5.0 mg/g of ointment. The concentration preferably ranges from 2.5 mg/g to 4.5 mg/g or 3.0 mg/g to 4.0 mg/g. More preferably it is about 3.5 mg/g.

The ointment optionally contains mineral oil and/or white petrolatum.

The quinolone antibiotic containing formulations are used to treat a variety of dermatological bacterial infections. Nonlimiting examples of such infections include: Folliculitis; Furuncles; Carbuncles; Erysipelas; Hydradenitis Supperativa; skin abcesses; Cellulitis; Paronychia; Pseudomonas Aeruginosa infections; and, Impetigo.

When used to treat a dermatological bacterial infection, either the aqueous solution or ointment described above is applied topically to a dermatological bacterial infection for at least three (3) days. Preferably, it is applied for at least five (5) or seven (7) days. More preferably, it is applied for at least ten (10) or twelve (12) days. Most preferably, it is applied for fourteen (14) days.

The amount of solution applied per application typically ranges from 0.01 mL to 1.0 mL. Preferably, the amount applied ranges from 0.05 mL to 0.90 mL or 0.1 mL to 0.80 mL. More preferably, the amount applied ranges from 0.2 mL to 0.7 mL.

The amount of ointment applied per application typically ranges from 0.01 g to 1.0 g. Preferably, the amount applied ranges from 0.05 g to 0.90 g or 0.1 g to 0.80 g. More preferably, the amount applied ranges from 0.2 g to 0.7 g.

The number of applications per day of either the solution or the ointment to the skin disorder are typically one (1) or two (2).

The aqueous solution or ointment including a quinolone antibiotic may be included in a kit. In that case, the kit further includes instructions on the use of the solution or ointment, which typically contain one or more of the following: the amount of solution or ointment used per application; the number of applications per day; and, the number of days for which the solution or ointment should be applied.

The quinolone antibiotic containing formulations may, if warranted, be sequentially applied topically to the skin of a patient exhibiting a dermatological bacterial infection, a burn, or a wound with a formulation containing one or more imidazole and/or triazole antifungal agents.

Where sequential antibiotic/antifungal treatment is deemed advisable, the aqueous solution or ointment may be included in a second type of kit. The kit, in addition to the solution or ointment, includes instructions on the use of the solution or ointment along with instructions on the sequential administration of a formulation containing one or more imidazole and/or triazole antifungal agents. The instructions typically include: the amount of quinolone containing solution or ointment to be used; the amount of imidazole and/or triazole antifungal agent containing formulation to be used; the number of sequential applications per day; and, the number of days for which the formulations should be sequentially applied.

The quinolone antibiotic containing formulations for topical application to the skin of a patient exhibiting a dermatological bacterial infection, a burn or a wound may also contain one or more imidazole and/or triazole antifungal agents in a suitable concentration.

1. A method of treating a dermatological bacterial infection, wherein the method includes the step of applying either an aqueous solution or ointment comprising Ciprofloxacin-HCl topically to the area of skin on a patient that is infected.

2. The method according to claim 1, wherein an aqueous solution is applied topically, and wherein the solution comprises a concentration of Ciprofloxacin-HCl ranging from 2 mg/mL of solution to 5 mg/mL of solution.

3. The method according to claim 1, wherein the ointment is applied topically, and wherein the ointment comprises a concentration of Ciprofloxacin-HCl ranging from 2.0 mg/g of ointment to 5.0 mg/g of ointment.

4. The method according to claim 2, wherein the solution is topically applied at least once per day for five days.

5. The method according to claim 3, wherein the ointment is topically applied at least once per day for five days.

6. The method according to claim 5, wherein the solution is sequentially applied with a formulation comprising an imidazole antifungal agent.

7. The method according to claim 5, wherein the ointment is sequentially applied with a formulation comprising an imidazole antifungal agent.
